(19) United States
(12) Patent Application Publication
(10) Pub. No.: US 2008/0045624 A1
(43) Pub. Date: Feb. 21, 2008

(54) Method For Producing Polymers By Dispersion Polymerization

(75) Inventors: Dennis Losch, Altrip (DE); Volker Seidl, Mannheim (DE); Pulakesh Mukherjee, Mannheim (DE); Hans-Ulrich Moritz, Bendesforf (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: BASF AKTIENSGESELLSCHAFT a German Corporation, Ludwigshafen (DE)

(21) Appl. No.: 11/660,631

(22) PCT Filed: Aug. 9, 2005
(86) PCT No.: PCT/EP05/08611

§ 371 Date: Feb. 20, 2007

(30) Foreign Application Priority Data
Sep. 2, 2004 (DE) 10 2004 042 946.4

(51) Int. Cl. C08F 2/10; A61L 15/24; C08F 226/10; B01J 20/26

(52) U.S. Cl. 522/167; 522/173; 526/201; 526/202

(57) ABSTRACT

Process for producing polymers by spray polymerization wherein the aqueous monomer solution comprises at least one displacer, so that the polymer separates from the aqueous solution during the polymerization; water soluble or water swellable compositions; and also their use for thickening and/or absorption of liquids.

The present invention relates to a process for producing polymers by spray polymerization, to water soluble or water swellable compositions and to their use for thickening and/or absorbing liquids.

Further embodiments of the present invention are discernible from the claims, the description and the examples. It will be appreciated that the hereinbefore identified and the hereinafter still to be more particularly described features of the subject matter of the present invention are utilizable not only in the particular combination indicated but also in other combinations without leaving the realm of the present invention.

U.S. Pat. No. 4,380,600, EP-A-0 183 466 and WO-A-97/34933 describe processes for producing aqueous dispersions of water soluble polymers having a high solids content. The polymer dispersions have a distinctly lower viscosity than polymer solutions of comparable solids content.

EP-A-0 398 151 describes the two stage production of polymeric thickeners. An aqueous polymer solution or dispersion is produced in a first stage by polymerization and spray dried in a second stage.

GB-A-0 777 306 describes the production of polymers by spray polymerization. The reaction is catalyzed by amides, such as acrylamide, acetamide and partially hydrolyzed polyacrylonitrile. The examples utilize 0.7% to 1.1% by weight, based on the monomer solution, of a polymerization catalyst. The polymers can also be used as thickeners for synthetic resin dispersions.

The present invention has for its object to provide an improved process for producing polymers and also water soluble and water swellable compositions which rapidly dissolve and rapidly swell, respectively, in aqueous fluids.

We have found that this object is achieved by a spray polymerization process comprising a monomer solution comprising

a) at least one water soluble ethylenically unsaturated monomer,

b) at least one initiator and

c) water,

being sprayed into a reaction space and polymerized, wherein the monomer solution further comprises at least one displacer d), the displacer being

d1) from 5% to 50% by weight of at least one water soluble polymer,

d2) from 1% to 40% by weight of at least one water soluble salt, all based on the monomer solution, or a mixture of displacers d1) and d2).

A displacer is a material, or a mixture of different materials, which is readily soluble in water and also reduces the solubility of the polymers obtainable by the present invention's process, in the aqueous solution.

The solubility of displacers is typically not less than 1 g/100 g of water, preferably not less than 5 g/100 g of water, more preferably not less than 25 g/100 g of water and most preferably not less than 50 g/100 g of water.

For example, the monomer solution comprising the displacer can be homogeneous. The resultant polymer is then insoluble, will separate in disperse form during the polymerization and continue to grow in the disperse phase.

But it is also possible for the monomers to be forced out of the aqueous phase by the displacer and for the polymerization to start in the disperse phase. In this case, the displacer is only added directly upstream of the reactor, so that there is no need for additives to stabilize the monomer dispersion.

Preferred displacers reduce the solubility of the polymer to such an extent at least that the polymer will form a disperse phase in the monomer solution when the monomer conversion is up to 90 mol %, preferably up to 50 mol % and more preferably up to 30 mol %.

Useful displacers include for example one water soluble polymer d1) or a mixture of two or more different water soluble polymers d1). Useful displacers further include one water soluble salt d2) or a mixture of two or more different water soluble salts d2). It is further possible for mixtures of water soluble polymers d1) and water soluble salts d2) to be used as a displacer.

Examples of water soluble polymers. d1) are graft polymers of vinyl acetate and/or vinyl propionate on polyethylene glycols; one- or bothsidedly alkyl-, carboxyl- or amino-endcapped polyethylene glycols; copolymers of alkylpolyalkylene glycol acrylates or alkylpolyalkylene glycol methacrylates and acrylic acid and/or methacrylic acid; polyalkylene glycols; one- or bothsidedly alkyl-, carboxyl- or amino-endcapped polyalkylene glycols; hydrolyzed copolymers of vinyl alkyl ethers and maleic anhydride in the form of the free carboxyl groups and in the form of the salts at least partially neutralized with alkali metal hydroxides or ammonium bases; and/or of a water soluble starch selected from the group consisting of cationically modified potato starch, anionically modified potato starch, degraded potato starch and maltodextrin; polyvinylpyrrolidones, poly-acrylic acids and polymethacrylic acids in the form of the free carboxyl groups and in the form of the salts at least partially neutralized with alkali metal hydroxides or ammonium bases; or a mixture of two or more of the aforementioned water soluble polymers. Very particular preference is given to polyvinylpyrrolidone, polyethylene glycol, polyvinylamine and/or polyacrylic acid.

The average molecular weight of the water soluble polymers is up to 250 000 g/mol, preferably up to 100 000 g/mol and more preferably up to 50 000 g/mol. Higher molecular weights increase the viscosity of the monomer solution and hence make spraying into the reaction space more difficult.

In principle it is also possible to use water soluble polymers d1) based on monomers a). This applies for example when the monomer solution to be used comprises polyacrylic acid displacer as well as acrylic acid monomer a).

The concentration of water soluble polymer d1) in the monomer solution is typically in the range from 5% to 50% by weight, preferably in the range from 8% to 30% by weight, and more preferably in the range from 10% to 20% by weight.

Water soluble salts d2) are preferably inorganic salts, examples being sodium sulfate, sodium chloride, potassium sulfate or potassium chloride, or a mixture of two or more thereof. Sodium sulfate is very particularly preferred.

The concentration of water soluble salt d2) in the monomer solution is typically in the range from 1% to 40% by weight, preferably in the range from 2% to 30% by weight and more preferably in the range from 5% to 20% by weight.

In a preferred embodiment, the monomer solution further comprises at least one complexing agent, preferably ethylenediaminetetraacetic acid. A complexing agent will for example complex iron ions dissolved out of pipework, and minimize any possible interaction of iron ions with the polymerization. This is important in particular because transition metal ions are used as catalysts in graft polymerizations. But a graft polymerization with polymer d1) displacer serving as a grafting base is unwanted in the process of the present invention.

The concentration of complexing agent in the monomer solution is typically in the range from 0.0001% to 0.1% by weight, preferably in the range from 0.0005% to 0.05% by weight and more preferably in the range from 0.001% to 0.01% by weight.

The viscosity of the monomer solution can be measured in accordance with German standard specification DIN 51562 and is up to 100 mPas, preferably up to 50 mPas, more preferably up to 25 mPas and most preferably up to 10 mPas at 23° C.

The reaction can be carried out in the presence of an inert carrier gas, in which case inert is to be understood as meaning that the carrier gas cannot react with the constituents of the monomer solution. It is also possible, of course, to use a mixture of different complexing agents. The inert gas is preferably nitrogen. The oxygen content of the inert carrier gas is advantageously below 1% by volume, preferably below 0.5% by volume and more preferably below 0.1% by volume.

The inert carrier gas can be led through the reaction space cocurrently with or counter-currently to the free-falling droplets of the monomer solution, preferably cocurrently.

The gas velocity is preferably such that flow in the reactor is laminar in that for example there are no convection eddies opposite to the general direction of flow, and is for example in the range from 0.02 to 1.5 m/s and preferably in the range from 0.05 to 0.4 m/s.

The reaction temperature is typically between 70 to 250° C., preferably 80 to 190° C. and more preferably 90 to 140° C.

The concentration of monomer a) in the monomer solution is typically in the range from 2% to 50% by weight, preferably in the range from 5% to 40% by weight and more preferably in the range from 10% to 30% by weight.

The solubility of monomer a) in water is typically not less than 1 g/100 g of water, preferably not less than 5 g/100 g water, more preferably not less than 25 g/100 g of water and most preferably not less than 50 g/100 g of water.

Ethylenically unsaturated monomers a) are for example ethylenically unsaturated C₃-C₆-carboxylic acids. These compounds are for example acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid and also the alkali metal or ammonium salts of these acids.

Further monomers a) are acrylamidopropanesulfonic acid, vinylphosphonic acid and/or alkali metal or ammonium salts of vinylsulfonic acid. Acids are used either in unneutralized form or in partially or 100% neutralized form.

Useful monomers a) further include monoethylenically unsaturated sulfonic or phosphonic acids, examples being allylsulfonic acid, sulfoethyl acrylate, sulfoethyl methacrylate, sulfopropyl acrylate, sulfopropyl methacrylate, 2-hydroxy-3-acryloyloxypropylsulfonic acid, 2-hydroxy-3-methacryloyloxypropylsulfonic acid, allylphosphonic acid, styrenesulfonic acid and 2-acrylamido-2-methylpropanesulfonic acid.

Further monomers a) are for example acrylamide, methacrylamide, crotonamide, acrylonitrile, methacrylonitrile, dimethylaminoethyl methacrylate, dimethylaminoethyl acrylate, dimethylaminopropyl acrylate, diethylaminopropyl acrylate, dimethylaminobutyl acrylate, dimethylaminoethyl methacrylate, diethylaminoethyl methacrylate, dimethylaminoneopentyl acrylate and dimethylaminoneopentyl methacrylate and also their quarternization products, for example with methyl chloride, hydroxyethyl acrylate, hydroxyethyl methacrylate, hydroxypropyl acrylate and hydroxypropyl methacrylate.

Further monomers a) are monomers which are obtainable by reaction of nitrogenous heterocycles and/or carboxamides, such as vinylimidazole, vinylpyrazole and also vinylpyrrolidone, vinylcaprolactam and vinylformamide, with acetylene and which can also be quaternized, for example with methyl chloride, and monomers obtainable by reaction of nitrogenous compounds, such as diallyldimethylammonium chloride, with allyl alcohol or allyl chloride.

It is further possible to use vinyl and allyl esters and also vinyl and allyl ethers, such as vinyl acetate, allyl acetate, methyl vinyl ether and methyl allyl ether as monomers a).

The monomers a) can be used alone or mixed with each or one another, for example mixtures comprising two or more monomers a). Preference is given to using mixtures comprising two different monomers a).

Preferred monomers a) are acrylic acid, methacrylic acid and also the alkali metal or ammonium salts of these acids, acrylamide, methacrylamide, acrylonitrile, methacrylonitrile, itaconic acid, vinylformamide, vinylpyrrolidone, vinylimidazole, quaternized vinylimidazole, vinyl acetate, sodium vinylsulfonate, vinylphosphonic acid, hydroxyethyl acrylate, hydroxyethyl methacrylate, 2-acrylamido-2-methylpropanesulfonic acid, diallyldimethylammonium chloride and also mixtures thereof.

The monomers a) are preferably stabilized with a commercially available polymerization inhibitor, more preferably with a polymerization inhibitor which only acts together with oxygen, an example being hydroquinone monomethyl ether.

Commercially available polymerization inhibitors are polymerization inhibitors which are used as storage stabilizers in the respective monomers for product safety reasons. Examples of such storage stabilizers are hydroquinone, hydroquinone monomethyl ether, 2,5-di-tert-butylhydroquinone and 2,6-di-tert-butyl-4-methylphenol.

Preferred polymerization inhibitors require dissolved oxygen for optimum performance. Therefore, the polymerization inhibitors can be freed of dissolved oxygen prior to polymerization by inertization, i.e., flowing an inert gas, preferably nitrogen, through them. The oxygen content of the monomer solution prior to polymerization is preferably lowered to less than 1 weight ppm and more preferably to less than 0.5 weight ppm.

The monomers a) are polymerized with each or one another in aqueous solution in the presence of initiators b).

The initiators b) are used in customary amounts, for example in amounts from 0.001% to 5% by weight and preferably from 0.01% to 1% by weight, based on the monomers to be polymerized.

Useful initiators b) include all compounds which disintegrate into free radicals under the polymerization conditions, examples being peroxides, hydroperoxides, hydrogen peroxide, persulfates, azo compounds and redox initiators. Preference is given to the use of water-soluble initiators. In some cases it is advantageous to use mixtures of various initiators, examples being mixtures of hydrogen peroxide and sodium or potassium peroxodisulfate. Mixtures of hydrogen peroxide and sodium peroxodisulfate can be used in any proportion.

Useful organic peroxides are for example acetylacetone peroxide, methyl ethyl ketone peroxide, tert-butyl hydroperoxide, cumene hydroperoxide, tert-amyl perpivalate, tert-butyl perpivalate, tert-butyl perneohexanoate, tert-butyl perisobutyrate, tert-butyl per-2-ethylhexanoate, tert-butyl perisononanoate, tert-butyl permaleate, tert-butyl perbenzoate, di(2-ethylhexyl)peroxydicarbonate, dicyclohexyl peroxydicarbonate, di(4-tert-butylcyclohexyl)peroxydicarbonate, dimyristil peroxydicarbonate, diacetyl peroxydicarbonate, allyl peresters, cumyl peroxyneodecanoate, tert-butyl per-3,5,5-trimethylhexanoate, acetylcyclohexylsulfonyl peroxide, dilauryl peroxide, dibenzoyl peroxide and tert-amyl perneodecanoate.

Preferred initiators b) are azo compounds, examples being 2,2′-azobisisobutyronitrile, 2,2′-azobis(2,4-dimethylvaleronitrile) and 2,2′-azobis(4-methoxy-2,4-dimethylvaleronitrile), especially water soluble azo initiators, examples being 2,2′-azobis{2-[1-(2-hydroxyethyl)-2-imidazolin-2-yl]propane}dihydrochloride, 2,2′-azobis-(2-amidinopropane)dihydrochloride, 2,2′-azobis[2-(2-imidazolin-2-yl)propane]dihydrochloride and 2,2′-azobis[2-(5-methyl-2-imidazolin-2-yl)propane]dihydrochloride. Very particular preference is given to 2,2′-azobis[2-(2-imidazolin-2-yl)propane]dihydrochloride and 2,2′-azobis[2-(5-methyl-2-imidazolin-2-yl)propane)dihydrochloride.

Redox initiators are also further preferred initiators b). In redox initiators, the oxidizing component is at least one of the peroxo compounds indicated above and the reducing component is for example ascorbic acid, glucose, sorbose, ammonium bisulfite, ammonium sulfite, ammonium thiosulfate, ammonium hyposulfite, ammonium pyrosulfite, ammonium sulfide, alkali metal bisulfite, alkali metal sulfite, alkali metal thiosulfate, alkali metal hyposulfite, alkali metal pyrosulfite, alkali metal sulfide or sodium hydroxymethylsulfoxylate. The reducing component in the redox catalyst is preferably ascorbic acid or sodium pyrosulfite. Based on the amount of monomers used in the polymerization, for example from 1×10⁻⁵ to 1 mol % is used of the reducing component of the re-dox catalyst.

It is particularly preferable to induce the polymerization through the action of high energy radiation, in which case it is customary to use photoinitiators as initiator b). Useful photoinitators include for example α-splitters, H-abstracting systems or else azides. Examples of such initiators are benzophenone derivatives such as Michler's ketone, phenanthrene derivatives, fluorene derivatives, anthraquinone derivatives, thioxanthone derivatives, coumarin derivatives, benzoin ethers and derivatives thereof, azo compounds, such as the free-radical formers mentioned above, substituted hexaarylbisimidazoles or acylphosphine oxides, especially 2-hydroxy-2-methylpropiophenone (Darocure® 1173). Examples of azides are 2-(N,N-dimethylamino)ethyl 4-azidocinnamate, 2-(N,N-dimethylamino)ethyl 4-azidonaphthyl ketone, 2-(N,N-dimethylamino)ethyl 4-azidobenzoate, 5-azido-1-naphthyl 2′-(N,N-dimethylamino)ethyl sulfone, N-(4-sulfonylazidophenyl)maleimide, N-acetyl-4-sulfonylazidoaniline, 4-sulfonylazidoaniline, 4-azidoaniline, 4-azidophenacyl bromide, p-azidobenzoic acid, 2,6-bis(p-azidobenzylidene)cyclohexanone and 2,6-bis(p-azidobenzylidene)-4-methylcyclohexanone.

Particularly preferred initiators b) are azo initiators, such as 2,2′-azobis[2-(2-imidazolin-2-yl)propane]dihydrochloride and 2,2′-azobis[2-(5-methyl-2-imidazolin-2-yl)propane]dihydrochloride, and photoinitiators, such as 2-hydroxy-2-methylpropiophenone and 1-[4-(2-hydroxyethoxy)phenyl]-2-hydroxy-2-methyl-1-propan-1-one, redox initiators, such as sodium persulfate/hydroxymethylsulfinic acid, ammonium peroxodisulfate/hydroxymethylsulfinic acid, hydrogen peroxide/hydroxymethylsulfinic acid, sodium persulfate/ascorbic acid, ammonium peroxodisulfate/ascorbic acid and hydrogen peroxide/ascorbic acid, photoinitiators, such as 1-[4-(2-hydroxyethoxy)phenyl]-2-hydroxy-2-methyl-1-propan-1-one, and also mixtures thereof.

The monomer solution comprises water as component c).

The pH of the monomer solution is not decisive. But, to meet product requirements, the pH of the polymer of the present invention can be adjusted to the desired range via the pH of the monomer solution. Polymers for cosmetic applications, for example, should have a pH of around 7.

The polymerization of the monomers a) can be carried out in the presence of a crosslinker or of a combination of various crosslinkers. Crosslinkers are compounds having two or more polymerizable groups. Polymerization in the presence of at least one crosslinker is preferred.

The concentration of crosslinker in the monomer solution is typically in the range from 0.001% to 1% by weight and preferably in the range from 0.01% to 0.5% by weight.

Suitable crosslinkers are for example (meth)acrylic esters of polyhydric alcohols which may have been alkoxylated with up to 100 and usually up to 50 ethylene oxide and/or propylene oxide units. Suitable polyhydric alcohols are in particular C₂-C₁₀-alkanepolyols having 2 to 6 hydroxyl groups, such as ethylene glycol, glycerol, trimethylolpropane, pentaerythritol or sorbitol. Preferred crosslinkers are polyethylene glycol diacrylate and polyethylene glycol dimethacrylates, which are each derived from polyethylene glycols (which may be considered as ethoxylated ethylene glycol) having a molecular weight in the range from 200 to 2000. Further usable crosslinkers are methylenebisacrylamide, trimethylolpropane triacrylate, trimethylolpropane trimethacrylate, ethylene glycol diacrylate, propylene glycol diacrylate, butanediol diacrylate, hexanediol diacrylate, hexanediol dimethacrylate or diacrylates and dimethacrylates of block co-polymers formed from ethylene oxide and propylene oxide.

Useful crosslinkers further include diallyl carbonate, allyl carbonates or allyl ethers of polyhydric alcohols which may have been alkoxylated with up to 100, and usually up to 50 ethylene oxide and/or propylene oxide units, and allyl esters of polybasic carboxylic acids.

Allyl carbonates of polyhydric alcohols conform to the general formula I 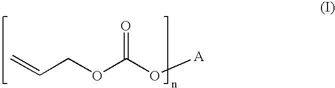
where A is the radical of a polyhydric alcohol which may have been alkoxylated with 0 to 100 and usually 0 to 50 ethylene oxide and/or propylene oxide units; and n represents the hydricness of the alcohol, for example an integer from 2 to 10 and preferably from 2 to 5. A particularly preferred example of such a compound is ethylene glycol di(allyl carbonate). Also suitable are particularly polyethylene glycol di(allyl carbonate)s which are derived from polyethylene glycols having a molecular weight in the range from 200 to 2000.

Preferred examples of allyl ethers are: polyethylene glycol diallyl ethers which are derived from polyethylene glycols having a molecular weight from 200 to 2000; pentaerythritol triallyl ether or trimethylolpropane diallyl ether. It is further possible to use reaction products of ethylene glycol diglycidyl ether or polyethylene glycol glycidyl ether with 2 mol of allyl alcohol and/or pentaerythritol triallyl ether.

A suitable allyl ester of a polyfunctional carboxylic acid is for example diallyl phtalate. Preferred monomers a) or combinations of monomers a) are acrylamide/quaternized vinylimidazole, acrylamide/diallyldimethylammonium chloride, acrylamide/quaternized dimethylaminoethyl acrylate, vinylpyrrolidone/quaternized vinylimidazole, vinylpyrrolidone/diallydimethylammonium chloride, vinylpyrrolidone/quaternized dimethylaminoethyl acrylate and acrylic acid.

Preferred initiators b) or combinations of initiators b) are azo initiators and azo initiators/photoinitiators.

Preferred displacers d) or combinations of displacers d) are polyvinylpyrrolidone/polyethylene glycol, sodium sulfate/polyvinylamine, polyethylene glycol/acrylic acid-maleic acid copolymers and acrylic acid-maleic acid copolymers.

The reaction is preferably carried out in apparatuses which are also suitable for spray drying. Such reactors are described for example in K. Masters, Spray Drying Handbook, 5th Edition, Longman, 1991, pages 23 to 66.

One or more spray nozzles can be used the in the process of the present invention. Usable spray nozzles are not subject to any restriction. The liquid to be spray dispensed may be fed to such nozzles under pressure. The atomizing of the liquid to be spray dispensed may in this case be effected by decompressing the liquid in the nozzle bore after the liquid has reached a certain minimum velocity. Also useful for the purposes of present invention are one-material nozzles, for example slot nozzles or swirl or whirl chambers (full cone nozzles, available for example from Düsen-Schlick GmbH, Germany, or from Spraying Systems Deutschland GmbH, Germany).

Preference for the purposes of the present invention is given to full cone nozzles. Of these, those having a spray cone opening angle in the range from 60 to 180° are preferred and those having an opening angle in the range from 90 to 120° are particularly preferred. For the purposes of the present invention, the average droplet diameter which results on spraying is typically less than 1000 μm, preferably less than 200 μm, more preferably less than 100 μm and customarily greater than 10 μm, preferably greater than 20 μm and more preferably greater than 50 μm, and can be determined by customary methods, such as light scattering, or by reference to the characteristic curves available from nozzle makers. The throughput per spray nozzle is advantageously in the range from 0.1 to 10 m³/h and frequently in the range from 0.5 to 5 m³/h.

The droplet diameter resulting in the course of spraying is typically in the range from 10 to 1 000 μm and preferably in the range from 50 to 500 μm.

The reaction can also be carried out in apparatuses in which the monomer solution can free fall in the form of monodisperse droplets. Suitable for this purpose are apparatuses as described for example in U.S. Pat. No. 5,269,980 column 3 lines 25 to 32.

Droplet formation through laminar jet disintegration as described in Rev. Sci. Instr., volume 38 (1966), pages 502 to 506 is likewise possible.

The use of spray nozzles for droplet generation is preferred.

The reaction space of the polymerization reactor can be carried out in overpressure or in underpressure, an underpressure of up to 100 mbar below ambient being preferred.

The polymerization reactor preferably has a carrier gas flowing through it. Cocurrent operation is preferred; that is, the carrier gas flows downwardly through the polymerization reactor.

The polymerization rate and the drying rate typically have different temperature dependencies. This can mean, for example, that the sprayed droplets dry before the desired conversion has been achieved. It is therefore advantageous to control the reaction rate and the drying rate separately.

The drying rate can be controlled via the water vapor content of the carrier gas. The water vapor content of the carrier gas is generally up to 90% by volume and preferably up to 50% by volume.

The polymerization rate can be controlled through the identity and amount of the initiator system used. The use of azo compounds or redox initiators as initiators b) is advantageous for controlling the polymerization rate. The lighting off characteristics of the polymerization are better controllable with azo compounds or redox initiators via the choice of initiator, initiator concentration and reaction temperature than for example with pure peroxide initiators.

Photoinitiators are particularly advantageous. When photoinitiators are used, the drying rate can be controlled to the desired value via the temperature without thereby significantly influencing the free-radical formation process at the same time.

The carrier gas is advantageously preheated to the reaction temperature of 70 to 250° C., preferably 80 to 190° C. and more preferably 90 to 140° C. upstream of the reactor.

The reaction offgas, i.e., the carrier gas leaving the reaction space, can be cooled down in a heat exchanger for example. Water and unconverted monomer condense in the process. Thereafter, the reaction offgas can be at least partially reheated and returned into the reactor as recycle gas. Preferably, the recycle gas is cooled down such that the cooled recycle gas has the water vapor fraction desired for the reaction. A portion of the reaction offgas can be removed from the system and replaced by fresh carrier gas, in which case unconverted monomers present in the reaction offgas can be separated off and recycled.

Particular preference is given to an integrated energy system whereby a portion of the heat rejected in the cooling of the reaction offgas is used to heat up the carrier gas.

The reaction space can be trace heated. Trace heating is adjusted such that the wall temperature is not less than 5° C. above reactor internal temperature and condensation at reactor walls is reliably avoided.

The reaction product is obtained in the form of free-flowing polymeric particles and can be removed from the reaction space in a conventional manner, preferably at the base via a conveying screw, and if appropriate be further dried to the desired residual moisture content and to the desired residual monomer content.

Preferably not less than 95% by weight of the particulate polymer has a particle diameter in the range from 5 to 1000 μm and preferably in the range from 10 to 500 μm, especially larger particle diameters being agglomerates of distinctly smaller primary particles. The particle size distribution can be determined by customary methods, for example analogously to recommended test method No. 420.2-02 “Particle Size Distribution-Sieve Fraction” of EDANA (European Disposables and Nonwovens Association).

The process of the present invention advantageously combines the production of a polymer dispersion with the drying of this dispersion in one step in which the heat of polymerization can simultaneously be used for drying. The displacer causes a polymer dispersion to form in the droplets which dries during the spray polymerization. Primary particles are formed which are distinctly smaller than the original droplets. In the course of drying in the reactor, these primary particles combine with the displacer to form agglomerates. This makes the polymers of the present invention readily redispersible and fast dissolving.

The present invention further provides water soluble or water swellable, pulverulent compositions comprising
i) at least 10% by weight, preferably not less than 30% by weight, more preferably not less than 50% by weight, of a polymerized water soluble ethylenically unsaturated monomer a) or of two or more copolymerized water soluble ethylenically unsaturated monomers a), and
ii) at least one displacer d), the displacer being
   ii1) at least 5% by weight, preferably at least 25% by weight and more preferably at least 35% by weight of at least one displacer d1),
   ii2) at least 2% by weight, preferably at least 10% by weight and more preferably at least 20% by weight of at least one displacer d2)
   all based on the composition, or
   ii3) a mixture of displacers d1) and d2),
   the average molecular weight of component ii) being lower than the average molecular weight of component i).
iii) if appropriate from 0.001% to 2% by weight, preferably from 0.01% to 1% by weight and more preferably from 0.02% to 0.5% by weight of at least one polymerized crosslinker,
   the sum total of the abovementioned components being not more than 100% by weight. Preferably not less than 95% by weight of the pulverulent composition has a particle diameter in the range from 5 to 1 000 μm and preferably from 10 to 500 μm.

The polymers or copolymers i) have average molecular weights of not less than 1 000 000 g/mol, preferably of not less than 2 500 000 g/mol and more preferably of not less than 5 000 000 g/mol.

The polymers ii) have average molecular weights of not more than 250 000 g/mol, preferably of not more than 100 000 g/mol and more preferably of not more than 50 000 g/mol.

In principle, the polymer i) and the polymers ii) can be constructed of the same monomers, but remain distinguishable because of their different average molecular weights (bimodal molecular weight distribution).

The compositions of the present invention comprise preferably less than 10% by weight of water, more preferably less than 5% by weight and most preferably less than 2% by weight.

The water soluble or water swellable polymers preparable by the process of the present invention are useful for thickening and/or absorption of fluids, especially aqueous systems, and also as absorbents in hygiene articles.

Uncrosslinked polymers dissolve in water, whereas crosslinked polymers swell substantially and likewise increase the viscosity of the aqueous medium substantially. For instance, 2% by weight aqueous solutions of polymers preparable by the process of the present invention have a pH 7 viscosity in the range from 800 to 40 000 mPas (measured in a Brookfield viscometer, spindle 6, at 20° C.), the viscosity increasing with the degree of crosslinking.

The polymers of the present invention are useful as thickeners for aqueous systems, examples being paper coating slips, pigment print pastes and waterborne coatings such as architectural coatings. They are also useful in cosmetics, examples being hair cosmetics such as conditioners or hair setting compositions or as thickeners for cosmetic formulations, for surface treatment of leather as well as for absorption of aqueous fluids.

The viscosity of 2% by weight aqueous solutions comprising polymers produced by the process of the present invention is not less than 500 mpas, preferably not less than 1 000 mPas and more preferably not less than 2 000 mPas at 23° C.

### EXAMPLES

### Example 1

9 kg of 30% by weight aqueous polyvinylpyrrolidone and 10 g of 40% by weight of aqueous ethylenediaminotetraacetic acid were dissolved in 27.5 kg of water. The mixture was admixed with 12 kg of Pluriol E1500 (polyethyleneglycol from BASF Aktiengesellschaft, Germany). The pH was adjusted to 6.75 with triethanolamine. 34.8 kg of 50% by weight aqueous acrylamide and 13.3 kg of 45% by weight aqueous methyl chloride quaternized vinylimidazole were metered in over 10 minutes. The mixture was then admixed with 30 g of Irgacure® 2959 (1-[4-(2-hydroxyethoxy)phenyl]-2-hydroxy-2-methyl-1-propan-1-one from Ciba Spezialitätenchemie, Switzerland) and 20 g of 2,2′-azobis(2-amidinopropane)dihydrochloride (V 50 Azo initiator from Wako Deutschland, Germany). This mixture was spray dispensed in a heated spray tower 8 m high and 2 m wide filled with nitrogen atmosphere (150° C., gas velocity 0.1 m/s in cocurrent). In the top third of the spray tower the droplets traveled past 6 iron-doped mercury UV lamps (6 kW each). A free flowing white powder was obtained at the base of the spray tower. The average particle size was 20 μm. This powder formed a clear solution in water. The 2% by weight solution had a pH of 7 and a viscosity of 1 050 mpas.

### Example 2

13.5 kg of 30% by weight aqueous polyvinylpyrrolidone and 10 g of 40% by weight of aqueous ethylenediaminotetraacetic acid were dissolved in 25.5 kg of water. The mixture was admixed with 10.5 kg of Pluriol E1500 (polyethyleneglycol from BASF Aktiengesellschaft, Germany). The pH was adjusted to 6.75 with triethanolamine. 34.8 kg of 50% by weight aqueous acrylamide and 7.3 kg of 60.7% by weight aqueous diallyldimethylammonium chloride were metered in over 10 minutes. The mixture was then admixed with 30 g of Irgacure® 2959 (1-[4-(2-hydroxyethoxy)phenyl]-2-hydroxy-2-methyl-1-propan-1-one from Ciba Spezialitätenchemie, Switzerland), 10 g of 2,2′-azobis(2-amidinopropane)dihydrochloride (V 50 Azo initiator from Wako Deutschland, Germany) and 10 g of ammonium peroxodisulfate. This mixture was spray dispensed in a heated spray tower 8 m high and 2 m wide filled with nitrogen atmosphere (150° C., gas velocity 0.1 m/s in cocurrent). In the top third of the spray tower the droplets traveled past 6 iron-doped mercury UV lamps (6 kW each). A free-flowing white powder was obtained at the base of the spray tower. The average particle size was 20 μm. This powder formed a clear solution in water. The 2% by weight solution had a pH of 7 and a viscosity of 1500 mPas.

### Example 3

9 kg of 30% by weight aqueous polyvinylpyrrolidone and 10 g of 40% by weight of aqueous ethylenediaminotetraacetic acid were dissolved in 27.5 kg of water. The mixture was admixed with 12 kg of Pluriol E1500 (polyethyleneglycol from BASF Aktiengesellschaft, Germany). The pH was adjusted to 6.75 with triethanolamine. 34.8 kg of 50% by weight aqueous acrylamide and 13.3 kg of 45% by weight aqueous methyl chloride-quaternized vinylimidazole and 37.3 kg of 98% by weight triallylamine were metered in over 10 minutes. The mixture was then admixed with 30 g of Irgacure® 2959 (1-[4-(2-hydroxyethoxy)phenyl]-2-hydroxy-2-methyl- 1-propan-1-one from Ciba Spezialitätenchemie, Switzerland) and 20 g of 2,2′-azobis(2-amidinopropane) dihydrochloride (V 50 Azo initiator from Wako Deutschland, Germany). This mixture was spray dispensed in a heated spray tower 8 m high and 2 m wide filled with nitrogen atmosphere (90° C., gas velocity 0.1 m/s in cocurrent). The monodisperse droplets were 400 μm in diameter. In the top third of the spray tower the droplets traveled past 6 iron-doped mercury UV lamps (6 kW each). A free-flowing white powder was obtained at the base of the spray tower. The average particle size was 220 μm. This powder formed a clear solution in water. The 2% by weight solution had a pH of 7 and a viscosity of 6 000 mPas.

### Example 4

13.5 kg of 30% by weight aqueous polyvinylpyrrolidone and 10 g of 40% by weight of aqueous ethylenediaminotetraacetic acid were dissolved in 16.7 kg of water. The mixture was admixed with 13.5 kg of Pluriol E1500 (polyethyleneglycol from BASF Aktiengesellschaft, Germany). The pH was adjusted to 6.75 with triethanolamine. 34.8 kg of 50% by weight aqueous acrylamide and 6.6 kg of 80% by weight aqueous methyl chloride quaternized dimethylaminoethyl acrylate were metered in over 10 minutes. The mixture was then admixed with 30 g of Irgacure® 2959 (1-[4-(2-hydroxyethoxy)phenyl]-2-hydroxy-2-methyl-1-propan-1-one from Ciba Spezialitätenchemie, Switzerland) and 20 g of 2,2′-azobis(2-amidinopropane) dihydrochloride (V 50 Azo initiator from Wako Deutschland, Germany). This mixture was spray dispensed in a heated spray tower 8 m high and 2 m wide filled with nitrogen atmosphere (90° C., gas velocity 0.1 m/s in cocurrent). The monodisperse droplets were 400 μm in diameter. In the top third of the spray tower the droplets traveled past 6 iron-doped mercury UV lamps (6 kW each). A free flowing white powder was obtained at the base of the spray tower. The average particle size was 170 μm. This powder formed a clear solution in water. The 2% by weight solution had a pH of 7 and a viscosity of 800 mPas.

### Example 5

7.7 kg of sodium sulfate, 12.8 kg of vinylpyrrolidone and 6.4 kg of 50% by weight aqueous methyl chloride quaternized vinylimidazole were dissolved in 30 kg of water. 7.5 kg of 20% by weight aqueous polyvinylamine (Catiofast® PR8106 from BASF Aktiengesellschaft, Germany) were added as a dispersant. The pH was adjusted to 6.75 with 5% by weight sulfuric acid. The mixture was then admixed with 50 g of 2,2′-azobis(2-amidinopropane)dihydrochloride (V 50 Azo initiator from Wako Deutschland, Germany). This mixture was spray dispensed in a heated spray tower 8 m high and 2 m wide filled with nitrogen atmosphere (150° C., gas velocity 0.1 m/s in cocurrent). A free flowing white powder was obtained at the base of the spray tower. The average particle size was 22 μm. This powder formed a clear solution in water. The 2% by weight solution had a pH of 7 and a viscosity of 2 000 mPas.

### Example 6

7.7 kg of sodium sulfate, 12.8 kg of vinylpyrrolidone, 5 kg of 65% by weight aqueous diallyldimethylammonium chloride and 48 g of pentaerythrityl tetraallyl ether were dissolved in 39 kg of water. 6.3 kg of 25% by weight aqueous polyvinylamine (Catiofast® PR8106 from BASF Aktiengesellschaft, Germany) were added as a dispersant. The pH was adjusted to 6.75 with 5% by weight sulfuric acid. The mixture was then admixed with 60 g of 2,2′-azobis(2-amidinopropane)dihydrochloride (V50 Azo initiator from Wako Deutschland, Germany). This mixture was spray dispensed in a heated spray tower 8 m high and 2 m wide filled with nitrogen atmosphere (150° C., gas velocity 0.1 m/s in cocurrent). A free flowing white powder was obtained at the base of the spray tower. The average particle size was 24 μm. This powder formed a clear solution in water. The 2% by weight solution had a pH of 7 and a viscosity of 4 000 mPas.

### Example 7

7.7 kg of sodium sulfate, 12.8 kg of vinylpyrrolidone, 6.4 kg of 45% by weight aqueous methyl chloride-quaternized vinylimidazole and 48 g of triallylamine were dissolved in 34.7 kg of water. 6.3 kg of Sokalan® PA80 (polyacrylic acid from BASF Aktiengesellschaft, Germany) were added as a dispersant. The pH was adjusted to 6.75 with 50% by weight sulfuric acid. The mixture was then admixed with 50 g of 2,2′-azobis(2-imidazolin-2-yl)propane]dihydrochloride (V44 Azo initiator from Wako Deutschland, Germany). This mixture was spray dispensed in a heated spray tower 8 m high and 2 m wide filled with nitrogen atmosphere (110° C., gas velocity 0.1 m/s in cocurrent). A free flowing white powder was obtained at the base of the spray tower. The average particle size was 20 μm. This powder formed a clear solution in water. The 2% by weight solution had a pH of 7 and a viscosity of 9 000 mPas.

### Example 8

9 kg of Sokalan® HP22 (polyacrylic acid from BASF Aktiengesellschaft, Germany) and 5.2 kg of Sokalan® CP2 (polyacrylic acid from BASF Aktiengesellschaft, Germany) were dissolved in 2.9 kg of water. 3 kg of acrylic acid were added within 10 minutes. The mixture was then admixed with 8 g of 2,2′-azobis[2-(2-imidazolin-2-yl)propane]dihydrochloride (V44 Azo initiator from Wako Deutschland, Germany). This mixture was spray dispensed in a heated spray tower 8 m high and 2 m wide filled with nitrogen atmosphere (110° C., gas velocity 0.1 m/s in cocurrent). A free flowing white powder was obtained at the base of the spray tower. The average particle size was 25 μm. This powder formed a clear solution in water. The 2% by weight solution had a pH of 7 and a viscosity of 5 000 mPas.

### Example 9

9 kg of Sokalan® HP22 (polyacrylic acid from BASF Aktiengesellschaft, Germany) and 5.2 kg of Sokalan® CP2 (polyacrylic acid from BASF Aktiengesellschaft, Germany) were dissolved in 2.9 kg of water. 3 kg of acrylic acid and 9 g of triallylamine were added within 10 minutes. The mixture was then admixed with 8 g of 2,2′-azobis[2-(2-imidazolin-2-yl)propane]dihydrochloride (V44 Azo initiator from Wako Deutschland, Germany). This mixture was spray dispensed in a heated spray tower 8 m high and 2 m wide filled with nitrogen atmosphere (110° C., gas velocity 0.1 m/s in cocurrent). A free flowing white powder was obtained at the base of the spray tower. The average particle size was 22 μm. This powder formed a clear solution in water. The 2% by weight solution had a pH of 7 and a viscosity of 40 000 mPas.

### Example 10

12 g of Pluriol® P600 (polypropyleneglycol from BASF Aktiengesellschaft, Germany) and 5.2 kg of Sokalan® CP2 (polyacrylic acid from BASF Aktiengesellschaft, Germany) were dissolved in 10.6 kg of water. 3 kg of acrylic acid were added within 10 minutes. The mixture was then admixed with 8 g of 2,2′-azobis[2-(2-imidazolin-2-yl)propane]dihydrochloride (V 44 Azo initiator from Wako Deutschland, Germany). This mixture was spray dispensed in a heated spray tower 8 m high and 2 m wide filled with nitrogen atmosphere (110° C., gas velocity 0.1 m/s in cocurrent). A free flowing white powder was obtained at the base of the spray tower. The average particle size was 22 μm. This powder formed a clear solution in water. The 2% by weight solution had a pH of 7 and a viscosity of 500 mpas.

1. A process for producing a polymer by spray polymerization comprising an aqueous monomer solution comprising
a) at least one water soluble ethylenically unsaturated monomer,
b) at least one initiators and
c) water,
being sprayed into a reaction space and polymerized, wherein the monomer Solution further comprises at least one displacer d), the displacer being
d1) from 5% to 50% by weight of at least one water soluble polymer,
d2) from 5% to 40% by weight of at least one water soluble salt,
all based on the monomer Solution, or a mixture of displacers d1) and d2).

2. The process according to claim 1 wherein the displacer d1) is polyvinylpyrrolidone, polyethylene glycol, polyvinylamine, polyacrylic acid, an acrylic acid-maleic acid copolymer, and/or a graft polymer of vinyl acetate and/or vinyl propionate on polyethylene glycol.

3. The process according to claim 1 wherein the displacer d2) is an inorganic salt.

4. The process according to claim 1 wherein the monomer a) is acrylic acid, vinylpyrrolidone, quaternized vinylimidazole, acrylamide, quaternized dimethylaminoethyl acrylate, and/or diallyldimethylammonium chloride.

5. The process according to claim 1 wherein the initiator b) is an azo compound, a redox initiator, and/or a photoinitiator.

6. The process according to claim 1 wherein the monomer solution further comprises a complexing agent.

7. The process according to claim 1 wherein the process is carried out in the presence of an inert carrier gas.

8. A water soluble or water swellable, pulverulent composition comprising
i) at least 10% by weight of a polymerized water soluble ethylenically unsaturated monomer a) or two or more copolymerized water soluble ethylenically unsaturated monomers a), and
ii) at least one displacer d), the displacer being
ii1) at least 25% by weight of at least one displacer d1),
ii2) at least 10% by weight of at least one inorganic displacer d2)
all based on the composition, or
ii3) a mixture of displacers d1) and d2),
an average molecular weight of component ii) being lower than the an average molecular weight of component i).

9. (canceled)

10. A thickened aqueous fluid comprising a composition according to claim 8 and water.

11. A method of thickening or absorbing an aqueous fluid comprising contacting the fluid with a composition of claim 8.
